(19) United States
(12) Patent Application Publication
(10) Pub. No.: US 2004/0235029 A1
(43) Pub. Date: Nov. 25, 2004

(54) In vitro translation system

(76) Inventors: Jae Moon Lee, Cupertino, CA (US); Douglas Iwen Buckley, Woodside, CA (US); Michael Robert Cancilla, Millbrae, CA (US); Damian E. Curtis, Burlingame, CA (US); Krista K. Bowman, Redwood City, CA (US); Hangjun Zhan, Foster City, CA (US); Margie Ciancio, Havertown, PA (US)

Correspondence Address: PATENT DEPT, EXELIXIS, INC., 170 HARBOR WAY, P.O. BOX 511, SOUTH SAN FRANCISCO, CA, 94083-0511, US

(21) Appl. No.: 10/832,820

(22) Filed: Apr. 27, 2004

Related U.S. Application Data
(60) Provisional application No. 60/465,963, filed on Apr. 28, 2003.

(51) Int. Cl. C12Q 1/68; G01N 33/53

(52) U.S. Cl. 435/6; 435/7.1

(57) ABSTRACT

In vitro translation (IVT) systems and methods for increased expression of proteins from linear templates, using GamS, are provided. The proteins may be full length or protein fragments. The IVT system may be used in batch or continuous mode. The GamS may be used as GamS nucleic acid template, crude protein fraction, or purified protein product. The IVT system using GamS component may be employed in a high-throughput mode. The ability to predict expressible protein or fragments, and activity and solubility of a large-scale protein expression product based on the results obtained from high-throughput, small-scale IVT expression product is also provided.

### REFERENCE TO RELATED APPLICATIONS

This application claims priority to U.S. provisional patent application 60/465,963 filed Apr. 28, 2003. The contents of the prior application are hereby incorporated in their entirety.

### BACKGROUND OF THE INVENTION

In vitro translation (IVT), a cell-free method of protein expression, is an attractive alternative to the conventional in-vivo technologies for protein production such as bacterial fermentation and cell culture. Some advantages IVT has over cell-based systems are: 1) it allows direct access to reaction conditions; 2) it is free of all cell functions except protein production; 3) the products of the synthesis do not affect continued productivity; and 4) it is simpler, faster, and suitable for high-throughput expression systems. The nucleic acid that encodes the protein to be expressed is referred to as a “template”. Templates for IVT may be circular (inside plasmids, for example) or linear. Use of linear templates for IVT is advantageous over the use of circular templates, since linear templates can be made directly by PCR, thus avoiding many laborious steps such as subcloning, transformation, plasmid isolation, and sequencing. Therefore, IVT using linear templates is ideal for making a large number of different proteins in high-throughput mode as well as screening many different constructs or mutants of given genes. However, one drawback of IVT using linear templates is low protein yield when used in conjunction with E. coli extracts, mainly due to the degradation of linear DNA by exonuclease V, or ExoV of E. coli (see Pratt J M (1984) and references therein). ExoV, a component of RecBCD holoenzyme, harbors both ATP-dependent 3′- and 5′-exonuclease activities, and digests both single- and double-strand DNA. Several attempts have been made to improve the protein yield from linear templates by avoiding the ExoV activity. For example, ExoV mutant strains have been used to make extracts, however, those mutants grow poorly and extracts are contaminated with large amounts of host chromosomal DNA (Gold and Schweiger (1972); Jackson et al (1983); Yang et al (1980); Yu et al (2000)). As another example, temperature sensitive ExoV mutants have also been used such that extract is prepared at a temperature in which ExoV is active, and IVT reaction is done at a high temperature in which ExoV is inactive. Still, the limitation of the IVT reaction only at high temperature is a problem (Jackson et al (1983)). As yet another example, cell extracts have been fractionated to remove the exonuclease, however, the reproducibility and efficiency of quality of extract are problematic. Therefore, an improved IVT system with enhanced capability of producing protein from linear templates would be desirable for providing increased protein yield for research and drug discovery.

Bacteriophage lambda is known to carry a gene that inhibits the ExoV activity of a host cell. The gene, called “Gam” for gamma, is expressed at the late stage of the phage cycle and prevents its genomic linear DNA from degradation by ExoV before packaging into the phage particles (Karu et al (1975)). The Gam gene encodes a protein, referred to as “GamL”, which is 138 amino acids long and has a predicted molecular weight of 16,349 daltons. It has been purified from E. coli, and been shown to inhibit ExoV activity by binding directly to the enzyme, not DNA (Karu et al , supra). A shorter form of the Gam protein, referred to as “GamS” having the gam activity by genetic means has also been reported (Friedman and Hays (1986)). GamS lacks the N-terminal 40 amino acids due to translation initiation at an internal, in frame, ATG of the Gam gene. This results in the smaller GamS of 98 amino acids, and 11646 daltons. GamS exhibits all activities associated with a GamL protein in cells. However, to date, due to lack of purified GamS, it has not been determined which Gam protein (GamL, GamS, or both) is the functional protein having ExoV inhibition activity.

### SUMMARY OF THE INVENTION

The invention provides an in vitro translation (IVT) system for protein expression from linear templates comprising a GamS component. The GamS component may be in the form of a GamS-encoding nucleic acid, crude protein fraction, or purified protein product. Further, the IVT system may be employed in batch or continuous mode. The invention provides methods for increasing protein expression from linear templates in an IVT system comprising adding a GamS component into the system. The GamS component may be in the form of a GamS-encoding nucleic acid, crude protein fraction, or purified protein product. Further, the IVT system may be employed in batch or continuous mode.

The invention provides a high-throughput IVT system and method for increasing protein expression from an array of linear nucleic acid templates, with each nucleic acid template located in a well of a plurality of wells of a plate. The GamS component in this system and method is added to each well of the plate. The invention provides methods of identifying expressible proteins, and predicting protein solubility, activity, and expression in a large-scale protein expression system based on the results obtained from the high-throughput IVT system using GamS component.

The invention further provides kits for IVT for protein expression from linear templates, wherein the kits comprise a GamS component and one or more components necessary for carrying out IVT reactions.

### DETAILED DESCRIPTION OF THE INVENTION

The invention provides an in vitro transcription/translation (IVT) system and method for linear templates comprising a GamS component. The GamS component may be in the form of a GamS-encoding nucleic acid or protein. The IVT system may operate in batch or continuous mode. The IVT system may be employed in a high-throughput manner to provide simultaneous protein expression from an array of linear templates. In various alternative embodiments, the expressed protein is a full-length protein, or a protein fragment, such as a protein domain or subdomain, or a fusion or chimeric protein, among others. GamS inhibits the ExoV activity of E. coli, thus dramatically increasing the yield of the expressed protein as compared with an IVT method or system that does not employ GamS. The utility of the invention is the increased yield of the expressed protein, which, in turn, is useful in protein research and drug discovery applications, such as parallel protein synthesis, optimization of expression constructs, functional testing of PCR generated mutations, expression of truncated proteins or protein fragments for epitope or functional domain mapping, full length protein and protein domain crystallization for structural biology applications, and expression of toxic gene products, among others. An unexpected additional utility of the invention is that results of protein expression in small quantities using GamS allow prediction of protein solubility and activity for large-scale expression of the same protein. Various alternative large-scale expression systems such as baculovirus, E. coli, IVT, and mammalian systems, among others, may be employed for large-scale protein productions. Thus, the invention additionally provides methods for alternating between various protein production methods when switching between a small-scale and a large-scale expression system.

IVT Systems

As used herein, “IVT system” or “IVT system for protein expression from linear templates” refers to at least one component or reagent that, when combined with a linear template encoding a polypeptide of interest, allow in vitro translation of the polypeptide. Such systems typically comprise a cell extract capable of supporting in vitro translation, an RNA-polymerase, ATP, GTP, CTP, UTP, and amino acids, among other things. The linear template is a DNA molecule comprising a gene encoding the desired polypeptide under the control of a promoter specific to the RNA polymerase.

The linear template may be transcribed as part of the IVT system, or prepared prior to additon to the IVT system. Transcription of DNA can occur in vivo or in vitro, from prokaryotic or eukaryotic cells or cell extracts, prior to in vitro translation. In vivo transcription systems are difficult to work with, since intact cells are used. In vitro transcription systems for both prokaryotic and eukaryotic systems are commercially available, and well known in the art. In vitro translation systems that are made from prokaryotic cells such as E. coli, or from eukaryotic cells such as rabbit reticulocyte and wheat germ, or from DNA sequences cloned into a vector containing an RNA polymerase promoter are also well known in the art (Zubay (1973); Pelham (1976); Roberts (1973); Krieg P (1984)).

Transcription and translation can also occur simultaneously in a coupled IVT system, wherein the linear template contains appropriate regulatory elements, such as the T7 promoter, ribosome binding site and T7 terminator, and the IVTsystem contains appropriate elements for both transcription and translation reactions. Such systems are also well known in the art, exist for both eukaryotic and prokaryotic applications, and can use both circular and linear templates (Pratt (1984); U.S. Pat. Nos. 5,895,753, 5,665,563, and 6,399,323, among others). Coupled IVT systems are also commercially available. One example is the RTS™ system (Rapid Translation System) of Roche Biochemicals (Germany) which uses E. coli extracts and employs continuous exchange cell-free system (CECF) and an improved energy-regeneration system (Kim (2001)). Other examples of commercially available IVT systems that can also be used in the invention include ProteinScript PRO™ of Ambion (Austin, Tex.), and TNT® system of Promega (Madison, Wis.), among others.

As used herein, IVT systems of the invention refer to systems wherein the transcription and translation reactions are carried out independently, as well as systems in which the transcription and translation reactions are carried out simultaneously (i.e. coupled systems).

IVT systems may operate in continuous mode or in batch mode. In a continuous mode IVT, the reaction products are continuously removed from the system, and the starting materials are continuously restored (continuous exchange cell-free system (CECF)) to improve the yield of the protein products (Spirin et al (1988), and U.S. Pat. No. 5,478,730). In contrast, batch mode IVT produces a limited quantity of protein, since the reaction products remain in the system, and the starting materials are not continuously introduced. Depending on the protein, the batch mode typically produces less than 1 milligram (mg) of protein, whereas the continuous mode can produce significantly greater quantities.

IVT systems may be high-throughput, where an array (i.e., at least two) of linear templates is processed simultaneously in multi-well reaction plates, where each nucleic acid template is in a well of the plate. The reaction plate has at least 2 wells, and typically has 12-, 24-, 96-, 384-, or 1536-wells; other sizes may also be used.

Cell Extracts

Cell extracts, which can be used for translation reactions alone, or for both transcription and translation reactions, must contain all the enzymes and factors to carry out the intended reactions, and in addition, be supplemented with amino acids, an energy regenerating component (e.g. ATP), and cofactors. Cell extracts for prokaryotic and eukaryotic IVT systems have been described, and are well-known in the art. Examples include prokaryotic lysates such as E. coli lysates, and eukaryotic lysates such as wheat germ extracts, insect cell lysates, rabbit reticulocyte lysates, rabbit oocyte lysates and human cell lysates (Zubay (1973), Pratt (1984), and U.S. Pat. No. 5,665,563, among others). Some of these extracts and lysates are available commercially (Promega; Madison, Wis.; Stratagene; La Jolla, Calif.; Amersham; Arlington Heights, Ill.; GIBCO/BRL; Grand Island, N.Y.).

Linear Template Production

Linear templates, which are the nucleic acid sequences from which the desired proteins are expressed, may be obtained using any available method. For instance, techniques for production of nucleic acids by using polymerase chain reaction (PCR), or nucleic acid synthesizers are well known in the art.

Linear templates may be designed such that the resulting protein may be expressed as a full-length protein or a protein fragment. Protein fragments include one or more protein domains or subdomains of the desired protein. Linear templates that encode mutated proteins can also be used. Linear templates may also be designed such that the resulting protein or protein fragment may be optionally expressed as a fusion, or chimeric protein product (i.e. it is joined via a peptide bond to a heterologous protein sequence of a different protein), for example to facilitate purification or detection. A chimeric product can be made by ligating the appropriate nucleic acid sequences encoding the desired amino acid sequences to each other using standard methods and expressing the chimeric product.

Detection of Protein Expression

Expression of the desired protein may be assayed based on the physical or functional properties of the protein (e.g. immunoassays, Western blotting, among others). Once a protein is obtained, it may be quantified and its activity measured by appropriate methods, such as immunoassay, bioassay, or other measurements of physical properties, such as crystallography.

Prediction of Protein Solubility and Activity Using GamS

We have discovered that the results obtained using high-throughput, small-scale IVT systems and methods using GamS as described above are predictors of activity, solubility, and expressibility of the same proteins produced at large-scale (i.e. typical yields of ≧1 mg of the protein product). For instance, constructs producing expressed, soluble, and/or active IVT products at high-throughput, small-scale setting, are predicted to produce active and soluble proteins in large-scale. Alternatively, constructs with insoluble and inactive IVT products in small-scale, high-throughput settings are less likely to produce soluble and active proteins in a large-scale setting. As such, the results obtained using high-throughput, small-scale IVT expression experiments can be used as predictors of proteins and protein fragments suitable for expression at any scale. The expressed protein products in high-throughput, small-scale IVT may be full-length proteins or protein fragments. Protein fragments include one or more protein domains, one or more protein subdomains, and fusion or chimeric proteins, among others. Thus, the IVT system of the invention serves as a predictor of protein or protein fragments suitable for expression at any scale. Prediction of expressible, active, or soluble proteins finds special applications for screens for small molecule modulators of the proteins, and structure assisted drug design, among other applications.

Alternative large-scale protein production systems include baculovirus systems, E. coli, IVT, and mammalian systems, among others. The switch from small-scale to large-scale protein expression provides the added advantage of the ability to switch from one protein expression system, such as IVT (cell-free), to another, such as baculovirus (cell-based).

An example of this utility and the switch from one system to another is provided in Example VII.

GamS Component

The invention provides IVT systems comprising a GamS component. The GamS component may be a GamS-encoding nucleic acid or protein, and may be provided in a variety of different forms. In one embodiment, the GamS component is provided as a crude protein extract, for example, as obtained from in vitro protein production or expression prior to purification. In an alternative embodiment, the GamS component is provided as a purified protein product. GamS proteins can be purified from natural sources, by standard methods (e.g. immunoaffinity purification). Methods for protein purification are well known in the art. GamS proteins can also be produced using IVT, as described further below. We have produced purified GamS protein (Example II), and further, provided data demonstrating GamS as the functional Gam protein (Example I). Typically, the GamS protein is added to the IVT system prior to the addition of the linear template encoding the protein of interest, to allow maximum exonuclease inhibition. Alternatively, GamS protein may be added along with or even after addition of the linear template to the IVT system. The effective amount of GamS protein, i.e., the amount that increases expression of proteins in an IVT system, for batch mode reactions, is in the range of 0.1 μg/ml to 10 μg/ml of GamS. A typical batch mode reaction is carried out in 50 μl of total volume, but the total volume may be as low as 15 μl. The effective amount of GamS for continuous IVT systems is in the range of 0.1 μg/ml to 100 μg/ml in a typical total of 1 ml to 10 ml of reaction volume.. Generally, the protein concentration of the E. coli extract is about 10 mg/ml in the reaction. The Gam protein of 2 μg/ml is about 0.2 μM, or 10 nmole in 50 μl. GamS concentrations of less than 0.1 μg/ml fail to produce significant effects, while GamS concentrations of more than 100 μg/ml may produce no further effects.

In an alternative embodiment, the GamS component is provided as a GamS-encoding nucleic acid for expression along with expression of the target protein (a process also known as co-expression) in an IVT system. GamS-encoding nucleic acids may be obtained as described in the Template production section. The amount of GamS in a typical co-expression experiment is determined based upon the protein target. Since co-expression of two or more different proteins can cause decreased expression of target protein due to competition for transcription and translation machinery, the optimum concentration of GamS template for the highest yield of target protein may be determined experimentally.

In an alternative embodiment, the GamS component is produced by the E. coli from which extracts are made. This method alleviates the need to introduce GamS externally. GamS of bacteriophage lambda shares significant sequence similarity and identity with a number of other Gam sequences, such as Gam protein of bacteriophage VT2-Sa (GI#9633411; SEQ ID NO:5), Gam of bacteriophage 933W (GI#9632481; SEQ ID NO:6), Gam of bacteriophage lysogen from Ecoli CFT037 (GI#26247406; SEQ ID NO:7), Gam of bacteriophage lysogen from Ecoli 0157:H7 (GI#7649836; SEQ ID NO:8), Gam of prophage CP-933V (GI#15802666; SEQ ID NO:9), Gam of bacteriophage lysogen from Shigella dysenteria (GI#6759958; SEQ ID NO:10), and Gam of bacteriophage lysogen from salmonella (GI#16759880; SEQ ID NO:11). These Gam genes and proteins can work effectively in the instant invention as alternatives to GamS component of bacteriophage lambda. Furthermore, other phage proteins with similar exonuclease inhibitory activity have also been described. These proteins exert their effect by directly binding to the DNA ends. While working with these proteins might prove difficult (since the protein needs to be added to the DNA, and might cause DNA aggregation, or interfere with the promoter located close to the ends of DNA), they can be used in place of GamS as well.

The invention provides kits for cell free protein expression from linear templates, where such kits include GamS and one or more components necessary for carrying out IVT reactions, where such components include enzymes, e.g. polymerases, reverse transcriptases, endonucleoses, dNTPs, buffers, and the like, and instructional material for carrying out the subject methodology. Such kits find use for production of enhanced quantities of proteins from nucleic acid templates.

All references cited herein, including patents, patent applications, and publications are incorporated in their entireties.

### EXAMPLES

The following experimental section and examples are offered by way of illustration and not by way of limitation.

I. Expression of GamL and GamS

GamS and GamL were expressed by IVT. PCR was employed to generate linear templates for GamL and GamS which also encoded C-terminal 6His tags, using the RTS™ Linear Template Kit of Roche Biochemicals (Germany). The primers used for the GamL were:

| | | |
|---|---|---|
| For 5′: | CTTTAAGAAG GAGATATACCATGGATATTAATACTGAAACTG | (SEQ ID NO:1) |
| For 3′: | ATGATGATGAGAACCCCCCCC TTATACCTCTGAATCAATATCA | (SEQ ID NO:2) |

The primers used for the GamS were:

| | | |
|---|---|---|
| For 5′: | CTTTAAGAAGGAGATATACCATGAACGCTTATTACATTCAGG | (SEQ ID NO:3) |
| For 3′: | ATGATGATGAGAACCCCCCCC TTATACCTCTGAATCAATATCA. | (SEQ ID NO:4) |

The generated linear templates were then subcloned into the plasmid vector pCAP (included in the cloning kit) by blunt end ligation using the PCR Cloning Kit of Roche Biochemicals (Germany).

Proteins were expressed using the RTS™ 100HY kit of Roche Biochemicals (Germany) in a batch mode from the plasmid templates, following manufacturer's protocols. The produced GamL and GamS proteins were analyzed by SDS-PAGE. GamL protein was expressed in the insoluble fraction. The calculated MW of GamL protein in the construct was 17,426 dalton. In contrast, the GamS protein was expressed in the soluble fraction, with a calculated MW of 12,724 dalton. The same results were obtained when the proteins were expressed directly from the linear templates. These data suggest the GamS protein as the functional form. For this reason, the GamS was chosen for further studies. GamL protein was also used as a control in the following studies, but failed to show any activity.

II. Purification of GamS

GamS protein was produced in a continuous IVT system using RTS™ 500HY of Roche Biochemicals, following the manufacturer's protocols from the GamS expression vector as described in Example I. The GamS protein was produced at more than 1 mg/ml in the soluble fraction. Pure protein was obtained after affinity purification through a nickel column (Qiagen) following the standard methods.

III. Effect of GamS on Production of Various Proteins via IVT

The purified GamS protein of Example II was added to the RTS™ 100HY reaction mixture (batch mode) containing the linear PCR template of the GFP to test the stimulatory activity of GamS protein. A linear template was made for the green fluorescent protein (GFP) with a C-terminal His tag and used as an example. The typical concentration of the GFP linear template was 2 to 5 μg/ml in the final reaction. Typically, the GamS protein was added to the reaction mixture and incubated for 20 minutes on ice before adding the GFP linear template. Since GamS binds and blocks ExoV, it was added into the reaction prior to addition of the DNA template. GamS might be added along with or even after addition of nucleic acid template, but in these cases some nucleic acid might be digested before ExoV inhibition activity of GamS, thus resulting in reduced yield of the resulting protein product. The following GamS concentrations were used in the experiments: 0.5, 1, 2, 5, and 10 μg/ml. Coomassie staining of the gel for reaction products indicated that GFP protein synthesis was increased notably for each GamS concentration as compared with control reactions lacking GamS, and was approximately three fold at 2 μg/ml of GamS. Concentrations larger than 2 μg/ml of GamS resulted in slight further increase in GFP protein synthesis. To confirm that GamS can increase the expression of proteins other than GFP, the GamS protein was tested on expression of three other proteins (protein kinases) from linear templates. The GamS was added at 2 μg/ml in these experiments, and increased protein expression for all of the proteins. These data clearly demonstrate that the GamS protein enhances protein yield in linear-template-based IVT for various proteins.

We next wanted to test the effect of GamS on protein expression in continuous mode IVT. In general, the preferred template in continuous mode IVT is circular, not linear, probably due to rapid degradation of the templates by continuous ATP supply, in addition to the exonuclease activity. However, linear templates were used for our studies. The RTS™ 500HY was used to express GFP from its PCR-template with the purified GamS protein (2 μg/ml). Reaction products were run on SDS-PAGE after 3 and 18 hours of incubation. Using the PCR-template alone, the GFP protein was expressed below detection limit by Coomassie staining of gels. However, inclusion of the GamS protein in the reaction dramatically increased GFP expression at both 3 and 18 hours of incubation. Overall, more than 1 mg/ml of GFP was obtained. These data indicate that using GamS protein allows the use of PCR-generated linear templates, instead of circular templates, in continuous IVT systems to produce high levels of proteins.

V. Effect of Crude GamS on Protein Expression in IVT

In order to determine whether the GamS in crude IVT product of Example I can have stimulation activity on protein production, the reactions in Example III were repeated using crude GamS instead of the purified GamS. One μl of the crude GamS protein from IVT reaction of Example I was added to 25 μl of reaction mixture, and incubated for 20 minutes on ice before adding 5 μg/ml of GFP linear template. PAGE (polyacrylamide gel electrophoresis) analysis of the IVT product followed by Coomassie staining of the gel showed a two fold increase of GFP compared with the control reaction without GamS. These results demonstrate that the crude GamS can be used without purification for stimulation of protein synthesis in IVT from linear templates.

V. Effect of Co-Expression of GamS on Protein Expression in IVT

A co-expression experiment was performed to test the stimulation of protein expression from linear templates directly using the GamS constructs without separate expression or purification of the GamS. The GFP linear template (5 μg/ml) was incubated with the GamS plasmid template (0.2 μg/ml) in the RTS™ 100HY system (Roche, Germany) in a batch mode. Coomassie staining of the gel of the reaction products indicated that co-expression of GamS caused a more than 2 fold increase in the expression of GFP. These data demonstrate that the GamS can be co-expressed for increasing protein synthesis in IVT from linear templates.

While the experiments presented here have employed GamS in association with commercial IVT products, it is important to note that any other commercial IVT product, or any non-commercial IVT system as explained in the instant specification can be substituted to produce the same results.

Taken together, these experiments indicate that GamS, via inhibition of the ExoV activity of E. coli, dramatically improves protein yields in IVT systems using linear templates as compared with systems lacking GamS. The IVT systems may be in batch or continuous mode. To enhance protein expression, GamS can be used as a co-expressed template, as a crude fraction, or as purified protein. Further, GamS may be used in any other systems that require protection from prokaryotic exonuclease activity.

VI. High-Throughput, Small-Scale IVT Using GamS

Detailed procedures for high-throughput IVT using an array of linear nucleic acid templates are described. Each template is placed in a well of a multi-well plate containing. all components necessary for IVT. For these experiments, we tested multiples of 8 constructs (48 constructs, for example) for each protein to assess solubility and/or activity. Each construct was chosen by selecting amino acid start and end positions corresponding to various domains of a protein, such as the kinase domains. To date we have tested the following combinations of forward and reverse primers: 3×16; 4×12; 8×6; 6×8; 12×4; and 16×3, and other combinations testing more constructs (e.g. 22×4 and 4×22) are also possible. Though this example employs 96 well plates, formats with less (such as 1, 6, 12, 24, 48, among others) or more wells (such as 384, 1536, and beyond) are expected to behave in the same manner. The average purification yield for each expression product for this type of high-throughput and small-scale experiment is the range of 0.5 to 1 μg.

1. Linear PCR-Templates for IVT

All automation steps are performed on Tecan and Hamilton robotic workstations.

A. 1st PCR.

This reaction is performed to define the amino acid boundaries on the nucleic acid template for protein expression. Use Roche Expand High Fidelity PCR (Cat. No. 1732 650) as follows: cDNA: 25-100 ng (QIAprep Spin Miniprep Kit, Qiagen Cat. No. 27106); 10× buffer (incl. Mg²⁺): 5.0 μl; dNTPs (25 mM): 0.4 μl; Gene-specific primers (10 μM): 2.0+2.0 μl; DMSO (100% v/v): 0.5 μl (recommended for human cDNA templates); High Fidelity Polymerase: 0.2 μl Pure H₂O to 50.0 μl.

Cycles:

1× 94° C. for 2 min;

20× 94° C. for 30 sec; 55° C. for 30 sec; 72° C. for 60 sec;

1× 72° C. for 2 min

Purify with Millipore Montage™ PCR μ96 (Cat. No. LSKMPCR50) 96-well filter units; resuspend in 25 μl pure water. Run 5 μl of PCR products on 1% (w/v) agarose E-gel (Invitrogen, Cat. No. G700801) to check yields.

B. 2nd PCR.

This reaction uses the product of the first reaction to produce more linear template for expression. Regulatory elements to perform IVT, and N-terminal HIS tags for purification are also added at this time.

Use Roche RTS™ 100 E. coli Linear Template Generation Set His6-tag (Cat. No. 3186 237) as follows: 1st PCR product: 2.0 μl (4.0 μl possible; 150-300 ng PCR1 template); 10× buffer (incl. Mg²⁺): 5.0 μl; dNTPs (25 mM): 0.4 μl; T7p primer 6 μM 4.0 μl; T7t primer 6 μM 4.0 μl; N-terminal His tag DNA 1.0 μl; DMSO (100% v/v): 0.5 μl; High Fidelity Polymerase: 0.2 μl; Pure H₂O to 50.0 μl.

Cycles:

1× 94° C. for 2 min;

25× 94° C. for 30 sec; 55° C. for 30 sec; 72° C. for 60 sec;

1× 72° C. for 2 min

Purify with Millipore Montage™ PCR μ96 (Cat. No. LSKMPCR50) 96-well filter units; resuspend in 25 μl pure water. Run 2 μl of PCR products on 1% (w/v) agarose E-gel (Invitrogen, Cat. No. G700801) to check yields. Need yield to be ≧50 ng/μl, expect PCRs on this gel to be over 100-bp bigger in size than 1st PCR.

2. IVT

Use: Roche, RTS™ 100 HY kit, Cat. No. 3168156.

A. Reagents

Thaw the Reconstitution buffer (Vial 5, all four).

Warm all other bottles to RT (four bottles for each of vial 1 to 3, one bottle for Vial 4).

Vial 1 (E. coli lysate): Reconstitute with 0.36 ml reconstitution buffer for each.

Vial 2 (Reaction mix): Reconstitute with 0.30 ml reconstitution buffer for each.

Vial 3 (Amino Acids): Reconstitute with 0.36 ml reconstitution buffer for each.

Vial 4 (Methionine): Reconstitute with 0.33 ml reconstitution buffer.

Store Vial #1 at −80° C. once dissolved.

All others can be stored at −20° C.

B. Reaction Mixtures for 2×96 Reactions (25 μl Final Vol)

| | 2× | 200× | |
|---|---|---|---|
| Vial 1: | 12.0 μl | × 100 = | 1.2 ml |
| Vial 2: | 10.0 μl | × 100 = | 1.0 ml |
| Vial 3: | 12.0 μl | × 100 = | 1.2 ml |
| Vial 4: | 1.0 μl | × 100 = | 0.1 ml |
| Vial 5: | 5.0 μl | × 100 = | 0.5 ml |
| | 40.0 μl | | 4.0 ml |

Add 5 μl of 10% Triton X-100 (a final 0.01% v/v).
Add 15 μl of GamS protein (0.68 mg/ml stock, a final
2 μg/ml).

C. IVT Reaction

Dispense 20 μl to each well.

Add≧150 ng DNA or a maximum of 5 μl purified 2nd PCR product.

Incubate at 30° C. for a minimum of 3 hours (shaking at 200 rpm).

Optional: keep 2.5 μl of reaction (store frozen) to estimate yields after purification.

3. Purification.

This step isolates IVT products based on their N-terminal HIS tags. Though this procedure has been optimized for purification of 6His-tagged proteins from 25 μl RTS™ reactions in 96-well plates, other purification methods and plate formats use variations of this same basic protocol. All steps are performed on Tecan robot.

A. Materials

MagneHis™ Protein Purification System (Promega, Cat. No. V8550).

B. Purification

1. Dispense 25 μl of MagneHis™ Beads (Promega).

2. Add 25 μl IVT reaction to Beads and mix.

3. Add 50 μl of MagneHis™ Wash Buffer to IVT and beads.

4. Place onto magnetic block (Promega).

5. Remove supernatant after mixing and 2 min RT incubation.

6. Wash three times with 150 μl of Wash Buffer supplied with Beads (Promega).

7. Elute with 50 μl of Elution Buffer supplied with kit (Promega).

8. Electrophoresis of 10 μl on SDS-PAGE and Western (optional).

9. Keep remaining 40 μl for activity assays.

4. SDS-Page.

Used to indicate yield and solubility.

A. Materials

Bio-Rad Criterion precast gel, 4-12%, 1.0 mm, 26 comb. 15 μl (Cat. No. 345-0034). Need 4 gels for 96 samples.

Bio-Rad 10× Tris/Glycine/SDS buffer (Cat. No. 161-0732).

Novex 4×SDS sample buffer (Cat. No. NP0007)

Bio-Rad Criterion Cell (for 2 gels).

Bio-Rad Criterion Dodeca Cell (up to 12 gels).

Adjustable multi pipettor for sample loading, such as 12 Channel IMPACT Equalizer® from Apogent Discoveries (Cat. No. 6230).

Pipette tips (30 μl) for the above pipettor, Cat. No. 7431.

Bio-Rad Biosafe Coomassie Blue G250 Stain (Cat. No. 161-0787).

B. Procedure

1. Mix 10 μl sample with 4 μl SDS sample buffer containing reducing agent in a 96 well plate.

2. Heat the sample at 90° C. for 5 min in a heating block.

3. Assemble the SDS gel in the gel tank (Need four gels for 96 samples).

4. Load the samples and run at 200 Volts (constant) for 40 min for a Criterion Cell. (For Criterion Dodeca Cell electrophoresis is performed at 4° C. for an hour or longer.)

5. Take the gels from the gel cassettes and transfer to staining trays containing 50% (v/v) ethanol and 10% (v/v) acetic acid (Note: label trays with the sample numbers). Leave them to fix overnight.

6. Wash twice the following morning for at least 30 minutes each in 50% (v/v) methanol and 5% (v/v) acetic acid.

7. Stain for at least 3 hrs in BioRad Biosafe Coomassie staining solution.

8. Destain once or twice with Washing solution for no more than 15 minutes each wash.

9. Transfer and destain in H₂O until the background is clear.

10. Dry and photograph gel.

5. Protein Assays.

At this stage, suitable assays for proteins of interest are conducted as explained above. An exemplary ATP consumption assay is provided here. This assay was employed in our experiments as a surrogate assay to measure kinase activity.

ATP Consumption Assay.

In this assay, the purified IVT product is incubated with substrate. Luciferase is then used to measure remaining ATP levels. These values are then compared to negative and positive control values.

A. Materials:

Greiner 384-well White Med Binding Plates (E&K,Cat. No. EK-30075).

Peptide/protein substrate mix: 20 mM Tris ph 7.5, 10 mM MgCl₂, 1 mM DTT, 0.02% Triton

X-100, 2 μM ATP, 10 μM Histone H1, 10 μM Casein, and 10 μM MBP.

Control active kinase made by IVT process.

Promega Kinase-Glo Luminescent Kinase Assay (Cat. No. V6712).

B. Assay Procedure:

1. Prepare ATP/peptide substrate mix (20 mM Tris ph 7.5, 10 mM MgCl₂, 10 mM DTT, 0.02% Triton X-100, 2 μM ATP, 10 μM Histone H1, 10 μM Casein, and 10 μM MBP).

2. Tecan Robot adds 20 μl ATP/substrate mix to assay plates (all wells).

3. Transfer 2 or 4 μl kinases from 96-well plate to the 384-well assay plate (four quadrants) using Tecan Robot. The kinase plate is formatted with negative controls (i.e., no kinase vector or kinase-dead mutant with all the common buffer components), and positive control (active kinase).

4. Mix and shake for 3 hrs for kinase reaction at ambient temperature.

5. Add 20 μl Kinase-Glo to entire plate.

6. Read plate on Wallac Victor multilabel reader.

7. Analyze data (% ATP consumption).

Characterization of 48 IVT Constructs for CAMK2G.

Calcium/calmodulin-dependent protein kinase II (CaM kinase II) is a ubiquitous serine/threonine protein kinase that has been implicated in diverse effects of hormones and neurotransmitters that utilize Ca2⁺ as a second messenger. The enzyme is an oligomeric protein composed of distinct but related subunits, alpha, beta, gamma, and delta, each encoded by a separate gene. Each subunit has alternatively spliced variants (Breen, M. A. and Ashcroft, S. J. H. (1997) FEBS Lett. 409: 375-379). Calcium/calmodulin-dependent protein kinase II Gamma (CAMK2G) may play a role in insulin secretion and growth control (Breen, M. A. and Ashcroft, S. J. H. (1997) Biochem Biophys Res Commun 236:473-8). Using the protocols provided above, we characterized 48 IVT constructs for CAMK2G (SEQ ID NO:12). We designed six forward PCR primers with start amino acid (AA) positions M1, T4, T6, T8, F10, T11, and eight reverse PCR primers with end AA positions V272, S276, S280, R284, K299, N313, G349, Q527. Forty-eight templates were amplified by covering all combinations of 6 forward and 8 reverse PCR primers. Following 48 IVT reactions with GamS, we performed SDS-PAGE and Western blot analyses of purified IVT proteins. High level protein expression (soluble bands on Coomassie stained gel and Western blot) was observed in constructs that contained the Auto Inhibitory Domain (AID; AA positions 285-299). We also performed kinase activity assays and detected the highest activities (near 100%ATP consumption) in constructs that lacked the AID, namely M1-S276; M1-S280; M1-R284; T4-S276; T4-S280; T4-R284; T6-S276; T6-S280; T6-R284; T8-S276; T8-S280; and T8-R284 constructs. These observations are consistent with the known biology of CAMK2G, in that the AID domain inhibits the activity of CAMK2G.

VII. Prediction of Protein Activity and Solubility

We have discovered that the results obtained using high-throughput, small-scale IVT expression experiments as described above are predictors of activity and solubility of the same proteins produced at large-scale. Further, we were able to transfer the protein products provided from a high-throughput, small-scale IVT experiment into a baculovirus expression vector system (BEVS) for large-scale production. As such, we were able to switch protein expression systems, in this case from a cell-free to a cell-based system of protein expression. Experimental details follow. We chose 24 IVT constructs representing 13 separate protein kinases that had produced soluble and/or active expression products in our high-throughput system.

1. Expression Subcloning.

The following protocol and descriptions detail the procedures for subcloning of expression products of a small-scale IVT into a baculovirus system.

Restriction Independent Cloning (RIC)

Primer Design

Design gene-specific primers using BamHI and EcoRI (+stop codon) overhang primers with these general criteria:

| | | | |
|---|---|---|---|
| Primer size | 12 | 20 | 35 |
| GC % | 20 | 40 | 90 |
| TM° C. | 55 | 60 | 65 |

Insert Preparation

1. RIC PCR (2 reactions per construct). Optional: perform triplicate to increase yields of insert DNAs: 10×Pfu buffer 5 μl; 10 mM dNTP 1.25 μl; Pfu polymerase 0.5 μl

2. 10 μM Phos primer 2 μl; 10 μM primer 2 μl; DNA (50 ng); H₂O quantity sufficient to total vol 50 μl.

3. Cycle at: 94° C. for 4 min, (94° C. for 45 sec, 55° C. for 30 sec, 72° C. for 4 min) repeat 29×, 72° C. for 5 min, 12° C.

4. Combine triplicate PCRs and purify PCR products using Qiagen's QIAquick PCR purification kit (Cat. No. 28104).

5. Denature and Anneal in 1×Pfu buffer.

6. Check yields of PCR reaction on agarose gels and quantify products. If necessary, excise ethidium bromide stained bands from the gel. Purify products using Qiagen's QIAquick gel extraction kit (Cat. No. 28704). For each construct, combine equal amounts of both PCR products (as estimated by gel or quantified using a spectrophotometer) to a total volume of 50 μl. Add 5 μl of Pfu buffer.

7. Cycling program: 95° C. for 4 min, ramp down at 0.1° C./sec to 15° C.

8. If a plasmid is the source of PCR template, treat with restriction endonuclease DpnI (New England Biolabs, Cat. No. R0176S). Purify annealed products using Qiagen's QIAquick PCR purification kit (Cat. No. 28104). Quantify these products by comparison with known DNA standards.

Ligation

Ligate BamHI/EcoRI digested vector at 25 ng/μl with purified, annealed insert at 40× excess with Roche Rapid DNA ligation kit (Cat. No. 1635379) according to the supplier's instructions. Vector is A5.2 BEVS cyto N-His-Tev (SEQ ID NO: 13).

$$ng insert = \frac{insert size ( kb )}{vector size ( kb )} ( 40 excess ) ( 25 ng vector )$$

Transformation

Add 2 μl ligation reaction to Invitrogen's One Shot® Top10 chemical competent cells (Cat. No. C4040-10 or C4040-50) and transform as per manufacturer's instructions. Plate 125 μl on each of 2 plates with appropriate antibiotics.

Colony Screening

Isolate DNA for 6-12 colonies per construct with Qiagen's QIAprep spin miniprep kit (cat. No 27104) or Qiagen's R.E.A.L. prep Kits (Cat. No.26171). Verify clones by end sequencing using vector specific primers, and if required, use internal gene-specific primers. Sequencing reaction: BigDye 2 μl; 5× sequencing buffer 2 μl; DMSO 0.5 μl; 2 μM primer 1 μl; DNA (50-100 ng) 1 μl; H₂O to final vol 10 μl.

Cycling Program: 94° C. for 4 min, (94° C. for 30 sec, 45° C. for 15 sec, 60° C. for 4 min) repeat 24×, 12° C., end. Use appropriate software to analyze sequence data in order to verify at least one clone per construct.

| | |
|---|---|
| BEVS forward sequencing primer | |
| 5′ TTCATACCGTCCCACCATCGGG 3′ | (SEQ ID NO:14) |
| BEVS reverse sequencing primer | |
| 5′ AAGAGAGTGAGTTTTTGGTTCTTGCC 3′ | (SEQ ID NO:15) |

Results

Using the above protocols, 24 IVT constructs were cloned by Restriction Independent Cloning (RIC). Using gene specific IVT generated PCR#1 or PCR#2 as template, a set of 4 gene specific BamHI and EcoRI(+stop codon) overhang primers were designed for each construct to amplify the desired gene domains. Denaturation and reannealing of the resulting PCR products produced a population of DNA in which 25% of the products contained the appropriate nucleotides to represent BamHI and EcoRI overhangs at the 5′ and 3′ ends of the cDNA, respectively. This DNA mixture for each of the 24 constructs was then ligated into A5.2 (SEQ ID NO:13), a modified pAcGP67 baculovirus DNA transfer vector (BD Pharmingen, Cat. No. 21223P) for baculovirus generation and cytoplasmic expression in Sf-9 insect cells. The DNA sequence of each of the resulting constructs was verified.

2. Baculovirus Stock Generation.

The following protocols and descriptions detail generation of baculovirus stock from subcloned expression products from step 1.

Co-Transfection Protocol Using Bacfectin from Clontech:

Baculogold Baculovirus DNA from BD Pharmingen, Cat. No. 554739.

1. Seed 1×10⁶ Sf9 cells into a 6-well culture plate. Incubate at 27° C. for 15-30 min. The plates should look 30-40% confluent.

2. Add into sterile microcentrifuge tubes in the following order:

| | |
|---|---|
| Sterile H₂O | 93.5-X μl |
| Plasmid DNA | X μl (final amount: |
| | 0.5-1 μg) |
| BaculoGold viral DNA (Cat. No. 554739) | 2.5 μl (0.1 mg/ml) |
| Bacfectin | 4 μl |
| Total | 100 μl |

Mix gently by tapping and quickly spin. Incubate at RT for 15 min to allow the Bacfectin to form complexes with the DNA.

3. Remove the media from the cells, and add 1.5 ml ESF-921 medium (Expression Systems LLC, Cat. No.96-001). The cells are ready for transfection.

4. Add the Bacfectin-DNA mixture dropwise to the medium while gently swirling the dish to mix. Incubate at 27° C. for 5 hr.

5. Add 1.5 ml TNM-FH Insect Medium (BD Pharmingen Cat. No.554760). Incubate at 27° C. for 5 days in a container with a wet paper towel to prevent evaporation.

For each transfection, set up:

a negative control well (Sf9 cells alone) to check normal cell growth

a positive control well: Biogreen or Wildtype (Wt) virus (add 5 μl of AcNPV Wild-Type High Titer virus provided to the positive control dish to check the health and infectability of the cells).

During a 5-day period, monitor the cell status by microscopic analysis, and check for any bacterial or yeast contamination. At day 3, the cells in the negative control dish should be confluent, while cells infected with WT virus should appear larger with enlarged nuclei and contain occlusion bodies.

6. After 5 days, collect the supernatant by spinning at 3000 rpm for 5 min, store the supernatant in 5-ml cryogenic vials wrapped with aluminum foil at 4° C. Take 300 μl of supernatant aliquot in a sterile microcentrifuge tube for the P1 to P2 amplification.

P1 to P2 Amplification

Mix equal volume of 2×10⁶ Sf9 cells/ml with pre-warmed TNM-FH Insect Medium in a sterile container (or a spinner flask) to a final cell density of 10⁶ cells/ml. Seed 3 ml cells in each well of 24-well plates, only in the 2 left and 2 right columns. Leave the middle two columns blank to avoid any contamination across the wells.

Add 30 μl P1 viral stock to each well. Each P1 stock is deposited into the 2 left or 2 right columns. Total vol is 24 ml per stock.

Incubate the 24-well plates in Hi-Gro (27° C., at 400 rpm). Check the plates daily to verify no bacterial or yeast contamination. Also before closing the chamber, wipe the top cover of the Hi-Gro chamber with KimwipeEX-L (Cat. No.34155), then with isopropyl alcohol wipes daily to keep dry and avoid contamination.

After 5 days, harvest the supernatant: spin at 3000 rpm for 5 min, and filter with 0.2 μm Millipore Steriflip filter (Cat. No.SCGP00525). Label the tubes with the harvest date, transfection code, and “P2 viral stock. Cover the tubes and store at 4° C. wrapped with Parafilm and aluminum foil.

For the repeat of virus amplification failed previously, count cells before harvesting, taking a note for the # of viable cells, the viability (%) as well as average cell diameter (μm).

Take out 2 aliquots of viral stocks before storage:

a. 100 μl (sterility not necessary) for Taqman™ titer determination, and quality control purposes

b. 1 ml (sterile) for −80° C. long-term storage

Titering BEVS Viral Stocks Using Taqman™ (Applied Biosystems)

Prepare viral DNA:

1. Add:

| | P1 | P2/P3 |
|---|---|---|
| Viral stock | 30 μl | 10 μ1 |
| Lysis buffer | 70 μl | 90 μl |
| Proteinase K (6 mg/ml) 1 μl | 1 μl | |
| Total | 100 μl | 100 μl |
| (include a wildtype control sample) | | |

2. 60° C., 1 h

3. 95° C., 10 min, cool to RT, spin briefly, then sit on ice till use.

4. Dilute viral DNA:

| | P1 | P2/P3 |
|---|---|---|
| | 1:15 | 1:50 |
| Treated Viral DNA | 2 μl | 2 μl |
| dH₂O | 28 μl | 98 μl |

5. For 96 well: in each well, complete in duplicate

$$\begin{matrix}Diluted viral DNA & 5 μl \\ Primers ( \#5448 + \#5449 ) 5 μM & 1 μl \\ d {H}_{2} O & 6.5 μl \\ 2 XTaqman SYGB Master Mix & 12.5 μl \\ Total & 25 μl\end{matrix} \} prepare a master mix$$

Include H₂O as a negative control and 7 standards

Primer Set:

pH.F3841 (#5448, SEQ ID NO:16)+pH.R3917 (#5449, SEQ ID NO:17)

| | |
|---|---|
| product: 77 bp | Tm = 68.08° C. |

6. Add 20 μl of master mix to each well, then add 5 μl of diluted viral DNA to the well

7. Cover the plate, quick spin (3000 rpm for 30 sec).

Reagents:

Lysis buffer: 10 mM Tris-HCl, pH 8.3, 100 μg/ml gelatin, 0.45% Triton X-100, 0.45% v/v Tween-20, 50 mM KCl, (store at 4° C.). Protease K: 6 mg/ml in dH₂O, (store at −20° C.).

8. Run samples on Taqman™ ABI PRISM, following manufacturer's standard protocols.

Results

Using the above protocols, each of the 24 constructs in the DNA transfer vector A5.2 was co-transfected into adherent Sf-9 insect cells cultured in ESF921 protein-free medium (Expression Systems, LLC, Woodland Calif. Cat. No. 96-001) at 27° C. with BaculoGold linearized viral DNA (BD Pharmigen Cat. No. 554739) and TNM-FH Insect Medium (BD Pharmingen Cat. No. 554760) according to the manufacturer's recommendations. The resulting P1 viral stocks were amplified twice to produce the P3 viral stocks to be used for large-scale protein production. Sf-9 cells cultured in suspension in ESF921 medium were infected at a cell density of 1×10⁶ cells/ml using an estimated multiplicity of infection (MOI) of 0.1 viral particles per cell and were harvested 3-5 days post infection. Sf-9 cells were removed by centrifugation, the resulting viral stocks were filtered to ensure sterility, and 3% heat-inactivated fetal bovine serum (FBS) was added for viral stability. All viral stocks were stored at 4° C. The titer of the P2 and P3 viral stocks was determined using a PCR-based Taqman analysis (ABI 7700, Applied Biosystems) to quantitate the number of viral genomes per volume of stock.

3. Production to Generate Biomass and P3 Viral Stock.

The following protocols and descriptions detail production of proteins from stocks generated in step 2 in insect culture fermentation runs.

1. Prepare 500 ml of log-phase Sf9 cells in a 2L unbaffled Erlenmeyer flask with a vented cap at a density of 2×10⁶ cells/ml.

2. Infect at MOI=0.5 with P2 Viral Stocks using the following calculation:

$$\begin{matrix}vol of viral stocks ( mL ) needed = \\ \frac{( MOI pfu \text{/} cell ) ( density of culture in cells \text{/} mL ) ( volume of culture in mL )}{titer of P2 Viral Stock in pfu \text{/} mL}\end{matrix}$$

3. Shake the flask at 130 rpm in a 27° C. incubator for 3 days. Monitor the progress of infection through counting cells. Take a note for cell density, cell viability (%), and average cell diameter (μm). Collect 1 ml cell cultures daily (day 1, day 2, and day 3): centrifuge at 3000 rpm for 1-5 min, aspirate the supernatant and store the cell pellets at −80° C. for assessing protein expression level. Collect an additional 1 ml sample at day 3 for quality control analysis.

4. For harvesting, transfer cell culture to 500 ml conical centrifuge bottles. Centrifuge at 3000 rpm (2600 r.c.f.) for 15 min at 4° C.

5. Filter supernatant with 0.2 μm PES blue-necked filter, add 3% heat inactivated FBS to supernatant for long-term storage. Label the bottles with the harvest date, transfection code, and “P3 viral stock”. Cover the bottles and store at 4° C. wrapped with Parafilm and aluminum foil.

6. Keep the cell pellet in the centrifuge bottles. Label the bottles with the harvest date, and transfection code. Store the pellets at −80° C., which are ready for purification.

7. Take out one aliquot of viral stocks before storage:

100 μl (sterility not required) for Taqman™ titer determination and quality control.

Western Blots for MOI Samples to Assess Protein Expression Level

Using Criterion XT Gels (4-12% Bis-Tris, BioRad Cat. No. 345-0125)

1. For each cell pellet from 1 ml day 3 sample, add 100 μl HIS lysis buffer (HIS lysis buffer: 50 mM Tris pH 8.0, 300 mM NaCl, 5 mM bME, 1% Triton X-100) to each pellet to resuspend cells.

2. Sonicate for 30 sec at maximum setting, ice, and repeat.

3. TOTAL FRACTION. Take out 10 μl of sonicated sample and add 70 μl 2× loading lysis buffer (80 mM Tris pH 7.5, 15% glycerol, 2% SDS, 100 mM DTT, 0.006% Bromophenol blue).

4. SOLUBLE FRACTION. Centrifuge sonicated sample at 14000 rpm, 4° C. for 10 minutes. Take out 10 μl of supernatant and add 70 μl 2× loading lysis buffer.

5. Boil 95° C. min.

6. Load 13 μl to gel (4-12% Criterion XT Bis-Tris gel). Load 40 ng of CTH-HADH (mix 2 μl 20 μng/μl stock with 2 μl 2× NuPAGE LDS sample buffer) to the same gel. Load 10 μl of SeeBlue Plus2 pre-stained protein standard on the same gel. Run the gel at 180V for 40 min (MES SDS Running Buffer)

7. Using Criterion Blotter with plate electrodes, transfer the gel with 1× NuPAGE Transfer buffer (+20% (v/v) methanol) at 100V for 30 min with PVDF membrane:

a. pre-wet PVDF membrane for 30 sec in 100% methanol.

b. briefly rinse in deionized water.

c. soak in 1× NuPAGE Transfer buffer (+20% methanol) for 3 minutes before use; presoak extra thick blot papers and filter papers briefly in the same transfer buffer immediately prior to use.

d. assemble the sandwich in the cassette on top of the black side: sponge-filter paper-gel-PVDF membrane-filter paper-sponge

8. Rinse the PVDF membrane with dH2O for 2×1 min.

9. Block the membrane with 3% BSA in TBS Buffer by rocking at RT for 30 minutes, or at 4° C. overnight if necessary.

10. Wash the membrane with TBS-T for 1×5 minutes, 2×2 minutes at room temperature.

11. Incubate the membrane with Penta His antibody (Cat. No. 34660) solution by gently rocking at RT for 1 hr.

12. (1:2000 dilution: 10 μl 0.2 mg/ml Penta His antibody stock in 20 ml TBS-T).

13. Wash the membrane with TBS-T for 3×5 minutes at room temperature.

14. Incubate the membrane with goat anti-mouse antibody (Pierce Cat. No. 31430) solution by gently rocking at RT for 1 hr. (1:5000 dilution: 5 μl anti-mouse antibody stock in 25 ml TBS-T).

15. Wash the membrane with TBS-T for 3×5 minutes followed by TBS wash for 5 minutes at room temperature.

16. Incubate the membrane with DAB solution by shaking for 1 min or till the stains reach the ideal level. (DAB solution: dissolve one tablet of DAB in 15 ml TBS, then add 15 μl of 30% hydrogen peroxide to the solution just before the use).

Purification of Expressed Products

1. Need Ni-NTA Superflow (Qiagen, Cat. No. 30430)

Roche Complete EDTA-free Protease Inhibitor cocktail tablets (Roche: 1-873-580)

PD-10 column (Amersham Biosciences)

Econo-pack disposable columns (25 ml) (Bio-Rad Cat. No. 732-1010)

| Lysis buffer: | Wash buffer: | Elution buffer: |
|---|---|---|
| 50 mM Tris pH 8.0 | 50 mM Tris pH 8.0 | 50 mM Tris pH 8.0 |
| 300 mM NaCl | 1 M NaCl | 300 mM NaCl |
| 5 mM βME | 5 mM βME | 5 mM βME |
| 10 mM Imdiazole | 15 mM Imidazole | 300 mM Imidazole |
| 1 mM Na Vanadate | | |

PIC-EDTA free (Roche, Cat. No.1-873-580)

Final Buffer:

50 mM Tris pH 8.0

300 mM NaCl

1 mM DTT

10% Glycerol

2. Dilute and fully resuspend biomass in 25 ml of lysis buffer.

3. Sonicate lysate for 30 sec, twice.

4. Spin down cell debris (˜120,000×g) for 45 sec.

5. Pack 0.5 ml of Ni-NTA resin into Econo-Pack gravity column and equilibrate the resin using a min. of 5 CV of lysis buffer.

6. Add clarified lysate to resin (1 passage).

7. Wash bound resin 2 CV of lysis buffer, follow by 10 CV of wash buffer.

8. Elute proteins with 2.5 ml of elution buffer into a PD10 column equilibrated in Final buffer.

9. Elute PD10 column with 3.5 ml of Final buffer and collect 3.5 ml.

10. Quantitate total protein of each protein by using Bradford Assay.

11. Run SDS-Page with 5 μl of sample per lane to check protein purity.

Results

Using the above protocols, each of the 24 constructs in the DNA transfer vector A5.2 was co-transfected into adherent Sf-9 insect cells cultured in ESF921 protein-free medium (Expression Systems, LLC, Woodland Calif.) at 27° C. with BaculoGold linearized viral DNA (BD Pharmigen) and TNM-FH Insect Medium (BD Pharmingen) according to manufacturer's recommendations. The resulting P1 viral stocks were amplified twice to produce the P3 viral stocks to be used for large-scale protein production. Sf-9 cells cultured in suspension in ESF921 medium were infected at a cell density of 1×10⁶ cells/ml using an estimated multiplicity of infection (MOI) of 0.1 viral particles per cell and were harvested 3-5 days post infection. Sf-9 cells were removed by centrifugation, the resulting viral stocks were filtered to ensure sterility, and 3% heat-inactivated fetal bovine serum (FBS) was added for viral stability. All viral stocks were stored at 4° C. The titer of the P2 and P3 viral stocks was determined using a PCR-based Taqman analysis (ABI 7700) to quantify the number of viral genomes per volume of stock.

Fifteen of the 24 constructs (63%) produced soluble protein and 7 of these 15 constructs (46%) produced active (substrate-dependent) protein products in a baculovirus expression system.

Taken together, these results demonstrate:

Ability to predict solubility and activity of a protein product from a large-scale protein production based on the outcome of a high-throughput, small-scale expression product of the same protein in an IVT system using GamS component; ability to predict expressible protein and protein fragments; and large-scale protein production in a baculovirus system using the products of a high throughput IVT system using a GamS component, thus switching between different protein expression methods.

References

Coligan J E et al, Current Protocols in Protein Science (eds.), John Wiley & Sons, New York, 1999.

Doonan S (ed.) Protein Purification Protocols, Humana Press, New Jersey, 1996.

Friedman, S A. and Hays, J. B. Selective Inhibition of Escherichia coli recBC activities by plasmid-encoded GamS function of phage lambda. Gene 43, 255-263, 1986.

Gold L M and Schweiger M., in Methods in Enzymology, Vol 20. Moldave K and Grossman L (eds), 1972.

Higgins S J and Hames B D (eds.) Protein Expression: A Practical Approach, Oxford University Press Inc., New York 1999.

Hunkapiller M, Kent S, Caruthers M, Dreyer W, Firca J, Giffin C, Horvath S, Hunkapiller T, Tempst P, Hood L. A microchemical facility for the analysis and synthesis of genes and proteins. Nature 1984 Jul. 12-18;310(5973):105-11.

Jackson M, Pratt J M, Holland I B. Enhanced polypeptide synthesis programmed by linear DNA fragments in cell-free extracts lacking exonuclease V. FEBS Lett. 1983 Nov. 14;163(2):221-4.

Karu, A. E., Sakaki, Y., Echols H., and Linn, S. The Gamma protein specified by bacteriophage lambda. Journal of Bacteriology. 250, 7377-7387, 1975.

Kim, D. M. and Swartz, J. R. Regeneration of adenosine triphosphate from glycolytic intermediates for cell-free protein synthesis. Biotechnology and Bioengineering 74, 309-316, 2001.

Krieg P A, Melton D A. Functional messenger RNAs are produced by SP6 in vitro transcription of cloned cDNAs. Nucleic Acids Res. 1984 Sep. 25;12(18):7057-70.

Pelham H R, Jackson R J. An efficient mRNA-dependent translation system from reticulocyte lysates. Eur J Biochem. 1976 Aug. 1;67(1):247-56.

Pratt, J. M. Coupled Transcription-Translation in Prokaryotic Cell-Free Systems. p179-209. In “Transcription and Translation: Practical approach” Edited by Hanes B. D. and Higgins, H. J. 1984.

Roberts B E, Paterson B M. Efficient translation of tobacco mosaic virus RNA and rabbit globin 9S RNA in a cell-free system from commercial wheat germ. Proc Natl Acad Sci USA. 1973 August;70(8):2330-4.

Spirin, A. S., Baranov V. I., Ryabova L. A., Ovodov S. Y., and Alakhov Y. B. A continuous cell-free translation system capable of producing polypeptides in high yield. Science 242, 1162-1164, 1988.

Stanbury P F et al., Principles of Fermentation Technology, 2^{nd} edition, Elsevier Science, New York, 1995.

Yang H L, Ivashkiv L, Chen H Z, Zubay G, Cashel M. Cell-free coupled transcription-translation system for investigation of linear DNA segments. Proc Natl Acad Sci USA. 1980 December;77(12):7029-33.

Yu D, Ellis H M, Lee E C, Jenkins N A, Copeland N G, Court D L. An efficient recombination system for chromosome engineering in Escherichia coli. Proc Natl Acad Sci USA. 2000 May 23;97(11):5978-83.

Zubay G. In vitro synthesis of protein in microbial systems. Annu Rev Genet. 1973;7:267-87.

What is claimed is:

1. An IVT system comprising:
a) at least one reagent necessary for protein expression from a linear template, and
b) a GamS component.

2. The system of claim 1 wherein the GamS component is a GamS-encoding nucleic acid template.

3. The system of claim 1 wherein the GamS component is GamS protein.

4. A method for increasing protein expression from a linear template in an IVT system comprising adding a GamS component into said system and performing steps necessary to express protein from the linear template.

5. The method of claim 4 wherein the GamS component is a GamS-encoding nucleic acid.

6. The method of claim 4 wherein the GamS component is a GamS protein.

7. The method of claim 4 wherein the IVT system is operated in batch mode.

8. The method of claim 4 wherein the IVT system is operated in continuous mode.

9. A high-throughput IVT system for protein expression from a linear template, comprising a GamS component.

10. A method for increasing protein expression from a plurality of linear templates in a high-throughput IVT system comprising adding a GamS component into said system and performing steps necessary to express protein from each nucleic acid template.

11. A method of predicting the activity and solubility of a desired protein product in a large-scale protein production comprising expressing the desired protein in a high-throughput IVT system using a GamS component, and determining the activity and solubility of the desired protein in the high-throughput IVT system, wherein it is predicted that the protein is active and soluble in a large-scale protein production if it is active and soluble in the high-throughput IVT system.

12. A method of predicting whether a desired protein or protein fragment is expressed in a large-scale protein expression system comprising expressing the desired protein or protein fragment in a high-throughput IVT using a GamS component; and determining whether the desired protein or fragment is expressed in the high-throughput IVT, wherein if the desired protein is expressed, it is predicted that the desired protein or fragment will be expressed in a large-scale protein expression system.

13. A kit for cell free protein expression from a linear template comprising a GamS component and one or more components necessary for carrying out IVT reactions.


SEQUENCE LISTING (17 sequences)

SEQ ID NO: 1 - DNA, 42 bp, Artificial
  5′ GamL primer
ctttaagaaggagatataccatggatattaatactgaaactg

SEQ ID NO: 2 - DNA, 43 bp, Artificial
  3′ GamL primer
atgatgatgagaaccccccccttatacctctgaatcaatatca

SEQ ID NO: 3 - DNA, 42 bp, Artificial
  5′ GamS Primer
ctttaagaaggagatataccatgaacgcttattacattcagg

SEQ ID NO: 4 - DNA, 43 bp, Artificial; identical to SEQ ID NO: 2
  3′ GamS primer

SEQ ID NO: 5 - protein, 138 aa, Bacteriophage VT2-SA
MDINTETEIKQKHSLTPFPVFLISPAFRGRYFHSYFRSSAMNAYYIQDRLEAQSWTRHYQQIAREEKEAELADDMGKGLPQHLFESLCIDHLQRHGASKKAITRAFDDDVEFQERMAEHTRYMVETIAHHQVDIDSEV

SEQ ID NO: 6 - protein, 98 aa, Bacteriophage 933W
MNAYYIQDRLEAQSWARHYQQIAREEKEAELADDMEKGLPQHLFESLCIDHLQRHGASKKAITRAFDDDVEFQERMAEHIRYMVETIAHHQVDIDSEV

SEQ ID NO: 7 - protein, 98 aa, Escherichia coli CFT073
MNAYYIQDRLEAQSWARHYQQIAREEKEAELADDMEKGLPQHLFESLCIDHLQRHGASKKAITRAFDDDVEFQERMAEHIRYIVETIAHHQADIDSEV

SEQ ID NO: 8 - protein, 98 aa, Escherichia coli 0157H7
MNAYYIQDRLEAQSWTRHYQQIAREEKEAELADDMGKGLPQHLFESLCIDHLQRHGASKKAITRAFDDDVEFQERMAEHIRYMVETIAHHQVDIDSEV

SEQ ID NO: 9 - protein, 98 aa, Escherichia coli 0157H7 EDL933
  misc_feature (9)..(9): Xaa can be any naturally occurring amino acid
MNAYYIQDXLEAQSWARYYQQIAREEKEAELADDMEKGLPQHLFESLCIDHLQRHGASKKAITRAFDDDVEFQERMAEHIRYMVETIAHHQVDIDSEV

SEQ ID NO: 10 - protein, 98 aa, Shigella dysenteriae
MNAWLIPDRIEEQSWARHYQQIAREETEAELADDLEKGLPQHLFESLCIDNLQRHGASKKAISRAFDDDVDFQERMAEHIRYMAETIARHQINIDSEV

SEQ ID NO: 11 - protein, 98 aa, salmonella enterica subsp. enterica serovar Typhi
MNAYLTYDRIEAQDWTRHYQQIAREEKESELADDLEKGLSLHMLESLCMDELPRHGANKKAISRAFDDDVEFQERASEFVRYMAETFSRHQIDIESEE

SEQ ID NO: 12 - protein, 518 aa, Homo sapiens
MATTATCTRFTDDYQLFEELGKGAFSVVRRCVKKTSTQEYAAKIINTKKLSARDHQKLEREARICRLLKHPNIVRLHDSISEEGFHYLVFDLVTGGELFEDIVAREYYSEADASHCIHQILESVNHIHQHDIVHRDLKPENLLLASKCKGAAVKLADFGLAIEVQGEQQAWFGFAGTPGYLSPEVLRKDPYGKPVDIWACGVILYILLVGYPPFWDEDQHKLYQQIKAGAYDFPSPEWDTVTPEAKNLINQMLTINPAKRITADQALKHPWVCQRSTVASMMHRQETVECLRKFNARRKLKGAILTTMLVSRNFSAAKSLLNKKSDGGVKPQSNNKNSLVSPAQEPAPLQTAMEPQTTVVHNATDGIKGSTESCNTTTEDEDLKVRKQEIIKITEQLIEAINNGDFEAYTKICDPGLTSFEPEALGNLVEGMDFHKFYFENLLSKNSKPIHTTILNPHVHVIGEDAACIAYIRLTQYIDGQGRPRTSQSEETRVWHRRDGKWLNVHYHCSGAPAAPLQ

SEQ ID NO: 13 - DNA, 9698 bp, Vector A5.2 BEVS cyto N-His-Tev
aagctttactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatattccggattattcataccgtcccaccatcgggcgcggatctatgctactaggatcgcatcaccatcaccatcacggtgaaaacctgtacttccagggatccaccgaattctgacaattccggagcggccgctgcagatctgatcctttcctgggacccggcaagaaccaaaaactcactctcttcaaggaaatccgtaatgttaaacccgacacgatgaagcttgtcgttggatggaaaggaaaagagttctacagggaaacttggacccgcttcatggaagacagcttccccattgttaacgaccaagaagtgatggatgttttccttgttgtcaacatgcgtcccactagacccaaccgttgttacaaattcctggcccaacacgctctgcgttgcgaccccgactatgtacctcatgacgtgattaggatcgtcgagccttcatgggtgggcagcaacaacgagtaccgcatcagcctggctaagaagggcggcggctgcccaataatgaaccttcactctgagtacaccaactcgttcgaacagttcatcgatcgtgtcatctgggagaacttctacaagcccatcgtttacatcggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggttcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 14 - DNA, 22 bp, Artificial
  BEVS forward sequencing primer
ttcataccgtcccaccatcggg

SEQ ID NO: 15 - DNA, 26 bp, Artificial
  BEVS reverse sequencing primer
aagagagtgagtttttggttcttgcc

SEQ ID NO: 16 - DNA, 24 bp, Artificial
  PH.F3841
ccaaaatgttgtacgaaccgttaa

SEQ ID NO: 17 - DNA, 22 bp, Artificial
  PH.R3917
caaacaactgctcgcagacaat